(19) United States
(12) Patent Application Publication
(10) Pub. No.: US 2006/0179495 A1
(43) Pub. Date: Aug. 10, 2006

(54) Tumor model with chromosomal rearrangement and uses thereof

(75) Inventor: Terence Howard Rabbitts, Cambridge (GB)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 11/255,618

(22) Filed: Oct. 21, 2005

Related U.S. Application Data
(63) Continuation of application No. PCT/GB04/01753, filed on Apr. 26, 2004.

(30) Foreign Application Priority Data
Apr. 24, 2003 (GB) 0309346.5

(51) Int. Cl. A01K 67/027

(52) U.S. Cl. 800/10; 800/18

(57) ABSTRACT

The invention relates to a method for generating a non-human animal model of a chromosomal rearrangement, comprising creating a transgenic non-human mammal expressing a site-specific recombinase under the control of a cell type specific promoter, and having sites recognized by the recombinase present in its genome such that a chromosomal rearrangement is catalysed by the recombinase.

The present invention relates to a model for tumourigenesis. In particular, the invention relates to an animal model for leukaemia which is based on a reciprocal chromosomal translocation which creates a fusion from the Mll and Enl genes.

### INTRODUCTION

The aetiology of tumours often involves chromosomal translocations in haematopoietic malignancies, sarcomas and epithelial tumours and these can either activate proto-oncogenes or create novel fusion genes which function in tumourigenesis (Rabbitts, 1994). These translocation products act at various levels in afflicted cells, although the translocation genes always encode intracellular proteins located in either the cytoplasm or nuclei. The recurrent chromosomal translocations in haematopoietic malignancies and in sarcomas generally display a tropism in respect of the cell type in which they are found, for instance the MLL gene fusion sub-types being restricted to either lymphoid or myeloid malignancies (Downing and Shannon, 2002; Look, 1997; Rowley, 1998). The MLL gene is a frequent site of chromosomal translocations in human leukaemias (Corral et al., 1993; Djabali et al., 1992; Domer et al., 1993; Gu et al., 1992; Thirman et al., 1993; Tkachuk et al., 1992), affecting over 30 different chromosomes and resulting in many leukaemia-associated MLL-gene fusions (Ayton and Cleary, 2001; Collins and Rabbitts, 2002). Different fusions distinguish distinct leukaemias, for instance the MLL-AF9 fusion is almost exclusively in myeloid leukaemias while MLL-AF4 is confined to B-lymphoid leukaemias (Gu et al., 1992; Rowley, 1998). Others, such as MLL-ENL can be found in myeloid and lymphoid leukaemias (Ayton and Cleary, 2001).

Mouse models which mimic the effects of chromosomal translocations, or which recapitulate these events, are essential to understand the determinants of frank malignancy and those dictating the restriction of translocations to specific lineages. In addition, with advancement of efforts to devise new reagents which can interfere with protein function, mouse models of human cancer are crucial to develop and test these rationally devised therapeutic approaches, prior to application in patients. Finally, mouse models of human cancer will be needed for in vivo testing of new compounds, which may have promising biochemical properties, in order to accelerate the translation of these into clinical use.

Mouse cancer models involving chromosomal translocation-genes have been either gain of function transgenic models (Adams et al., 1999; Pandolfi, 1998), retroviral transduction models or translocation mimics in which a gene fusion is created by homologous recombination into an endogenous target gene using embryonic stem cells (ES cells) (Corral et al., 1996). For instance, the consequences of MLL-associated translocations have been modelled in mice using retroviral transduction of bone marrow progenitors (Lavau et al., 1997; Schulte et al., 2002; Slany et al., 1998) or by homologous recombination knock-in of fusion genes (Corral et al., 1996; Dobson et al., 1999; Dobson et al., 2000). In the knock-in approach, homologous recombination was used to introduce fusion oncogenes into mice, via embryonic stem cells. In this way, a mouse model of the human MLL-AF9 gene fusion was made in which knock-in mice were found to develop an acute myeloid malignancy similar to that found in human patients with the chromosomal translocation t(9;11) (Corral et al., 1996; Dobson et al., 1999). While the knock-in approach is a close mimic of natural chromosomal translocations, it is limited by the fact that the homologous recombination event generates a knock-in of one allele which is subsequently transferred to all cells of the mouse. Thus the fusion gene will be expressed in all sites where the targeted gene is expressed, for instance as found for the Mll-AF9 knock-in fusion (Corral et al., 1996). In addition, dominant, embryonic lethal effects of knock-in alleles have been observed (Okuda et al., 1998; Yergeau et al., 1997). These can be overcome by creating conditional knock-in alleles such as the Amll-Eto example, which circumvents the embryonic lethal effects of the fusion, and causes AML to arise in mice (Higuchi et al., 2002).

The most ideal concept is the de novo creation of chromosomal translocations which could potentially be achieved by in vivo recombination systems, such as the Cre-loxP system (Smith et al., 1995; van Deursen et al., 1995).

SSRs of the integrase class such as FLP, from the 2μ episome of S. cereviseae, Cre, from the E. coli P1 phage, and R from Z. rouxii are able to mediate precise and conservative recombination between their binding sites. These proteins share overall similarity in their primary sequence and attract attention because they have been shown to function efficiently in a broad range of organisms from E. coli to mice. Their binding sites are 13bp inverted repeats which are sequence specifically recognised by a monomer per half site. Other SSRs having homologous structures are known.

The use of SSRs is potentially the nearest that could be achieved of a direct recapitulation of the de novo occurrence of human cancer-associated chromosomal translocations. The Cre-loxP recombination system has been shown to facilitate de novo chromosomal translocations in mice (Buchholz et al., 2000; Collins et al., 2000) but no tumour incidence was reported in these initial translocator mice.

Accordingly, despite the theoretical suitability of site-specific recombinases for replicating chromosomal translocation, it is uncertain whether Cre-lox mediated recombination can effectively recreate a tumour in an experimental animal.

### SUMMARY OF THE INVENTION

We have now established that reciprocal chromosomal translocations leading to tumour formation in experimental animals can be created using site-specific recombinases.

We have established a line of mice in which Cre recombinase is expressed via the haematopoietic Lmo2 gene (Warren et al., 1994) and where loxP sites have been engineered into Mll and Enl loci. We demonstrate that Cre-loxP-mediated inter-chromosomal recombination between the Mll and Enl genes creates reciprocal chromosomal translocations, which rapidly cause myeloid tumours. The rapid onset and high penetrance of leukaemogenesis suggests that the Mll-Enl translocation is sufficient to cause myeloid leukaemias without additional genetic changes, suggesting that human MLL translocations can cause cancer in the absence of secondary mutations (Ford et al., 1993). This approach is a direct recapitulation of human cancer-associated translocations, formally showing that these cause cancers, which are most likely clonal in origin. The strategy can be used to generate de novo reciprocal translocations that effectively recapitulate any naturally occurring translocation in human cancers.

In a first aspect of the invention, therefore, there is provided a method for generating a non-human animal model of a chromosomal rearrangement, comprising creating a transgenic non-human mammal expressing a site-specific recombinase under the control of a cell type-specific promoter, and having sites recognised by the recombinase present in its genome such that a chromosomal rearrangement is catalysed by the recombinase.

The chromosomal rearrangement may be any rearrangement, for example an insertion, deletion, inversion or translocation; advantageously, the chromosomal translocation is a reciprocal translocation.

The chromosomal rearrangement is preferably tumourigenic, that is, gives rise to tumours. The invention thus provides non-human animal models of tumour conditions associated with chromosomal rearrangements.

The invention is advantageously applied to the generation of models of tumours which are associated with chromosomal translocations. Preferably, the tumour is a haematopoietic tumour. Many proliferative and other conditions are known to be associated with chromosomal translocations, which can give rise to rearrangement, deletion or duplication of DNA. Such conditions are advantageously replicated by the method of the present invention.

Preferably, the cell type-specific promoter is the lmo2 promoter. The lmo2 gene is known in the art; see for example GenBank gi:7671618; gi:9799067; and Boehm et al., Proc. Natl. Acad. Sci. U.S.A. 88 (10), 4367-4371 (1991).

Recombinases are known in the art and include Cre, Flp and R recombinases. The sequences of these polypeptides are known: for example, the sequence of Cre recombinase is given in GenBank GI:5690440 and GI:17016300; Flp recombinase is given in GI:173333 and GI:173335; R recombinase, from Zygosaccharomyces rouxii, is given in GenBank GI:5260.

The cognate sites for these recombinases are lox (or loxP) sites for Cre, frt sites for Flp and Rs sites for R. Any recombinase system may be used in the invention; advantageously, it is selected from Cre-lox, Flp-frt and R-Rs.

Leukaemias which can be modelled by the method of the invention include all those which involve a chromosomal rearrangement; such rearrangements are very common in leukaemias. In particular, the human Mll gene is involved in a large number of leukaemias, and Mll-Enl fusions are found in myeloid and lymphoid leukaemias, as noted above.

In a further aspect, the invention provides a non-human animal model of chromosomal rearrangement, said animal expressing a site-specific recombinase under the control of a cell type-specific promoter. Sites recognised by the recombinase are inserted into the genome of the animal in positions such that recombination between the sites will give rise to the desired rearrangement. Rearrangements include deletions, inversions, duplications and both reciprocal and non-reciprocal translocations. Advantageously, the rearrangement is a translocation, preferably a reciprocal translocation, in which parts of non-homologous chromosomes exchange positions in the rearrangement.

The recombinase, which is advantageously Cre, Flp or R as described above, is placed under the control of a cell type-specific promoter which is advantageously the lmo2 promoter.

Advantageously, the reciprocal translocation gives rise to a Mll-Enl fusion. Thus, the transgenic animal according to the invention advantageously possesses a Mll-LoxP; Enl-LoxP; Cre phenotype. Alternatives which would be apparent to one skilled in the art include Mll-frt; Enl-frt; Flp and Mll-Rs; Enl-Rs; R. In a further embodiment, other translocation partners for Mll may also be used, replacing Enl.

The animal model according to the invention advantageously has leukaemia (or another tumour). It has surprisingly been found that Mll-LoxP; Enl-LoxP; Cre mice develop leukaemia with a high penetrance (approaching 100%) and rapid onset. Moreover, it appears that the creation of the Mll-Enl fusion is sufficient to give rise to the tumour in absence of any secondary mutations or other genetic changes. The tumour type developed by Mll-LoxP; Enl-LoxP; Cre mice is a myeloid leukaemia with a Mac-1; Gr-1 phenotype, as seen in human patients with leukaemia involving an Mll-Enl fusion.

Animal models in accordance with the invention are useful in testing anti-tumour therapeutics, or compounds with a potential anti-tumour effect. The rapid onset and high penetrance of the model in accordance with the invention is highly advantageous in testing therapeutic compounds, providing a more rapid and reliable result than the models available in the prior art. Compounds may be tested for effectiveness against any type of tumour, including leukeamias but also epithelial and other tumours which involve a chromosomal rearrangement.

In a further aspect, therefore, the invention provides a method for testing anti-tumour compounds, comprising exposing an non-human animal model in accordance with the present invention to said compounds and assessing the incidence of tumours in said model.

### DESCRIPTION OF THE FIGURES

FIG. 1. Targeting and Recombination Strategy for Mll and Enl Genes

LoxP recombination sites were introduced into the mouse Mll (Collins et al., 2000) and Enl genes using gene targeting in ES cells. The targeted cells were injected into blastocysts and chimaeric mice derived which carried the modified alleles. These were bred to obtain germ-line transmission and subsequently inter-bred with each other and with a line in which Cre recombinase is expressed from the Lmo2 gene.

A. Map of the mouse Mll gene indicating three exons of the gene and the BglII restriction site at which was cloned a loxP cassette with a hygromycin selectable marker (Johnson et al., 1995). The pMll-loxP-hygro targeting vector is shown in the middle line and the organisation of the targeted allele in the bottom line, showing that the loxP site is downstream of exon 10 of Mll.

B. The mouse Enl gene indicating exon 2 and the SphI restriction site at which was cloned a loxP cassette with a puromycin selectable marker (Linnell et al., 2001). The top line is the germ line map, the middle is the pEnl-loxP-puro targeting vector and the bottom is the structure of the targeted allele.

C. Cells or mice with both the Mll (chromosome 9) and Enl (chromosome 17) loxP alleles can undergo Cre-dependent inter-chromosomal reciprocal translocation. The derivative t(9.17) and t(17;9) are diagrammatically shown in the 3^{rd} and 4^{th} lines respectively.

D/E. To verify the ability of the Mll and Enl genes to participate in inter-chromosomal translocations, Cre recombinase was transiently expressed in ES cell lines carrying both loxP alleles. Genomic DNA or mRNA were prepared for genomic PCR (D) or RT-PCR (E) with Mll and Enl-specific primers. For the genomic PCR, primers MG1 (from Mll) and EG1 (from Enl) allowed amplification of a 430 bp fragment, the sequence of which comprised the junction of the Mll chromosome, the loxP site and the Enl chromosome (D). RT-PCR was carried out with RNA from the cells using an Mll exon 10 primer (MR1) and an Enl exon 2 primer (ER1) to determine if the Mll-Enl fusion mRNA could be detected. A 390 bp PCR was detected and sequenced, showing the in-frame fusion of Mll and Enl sequences (E).

FIG. 2. Myeloid Leukaemia Development Depends on Cre Expression

A cohort of animals (n=21) was generated which had the genotype Mll-loxP; Enl-loxP; Cre (Mll;Enl;Cre) and a control group (n=15) with Mll-loxP; Enl-loxP alleles (Mll;Enl) but lacking the Lmo2-Cre gene. Survival curves are shown in A. Mice were carefully observed from birth and post-mortem examination carried out at first signs of ill health. Biopsies of tissues were taken, together with blood smears. Smears were stained with MGG stain and photographs show the presence of a large number of leukocytes (B, X10) and high power shows immature myeloblasts and mature myeloid forms (B, X100). All organs examined were heavily infiltrated with these myeloid cells. Haematoxylin and eosin stained sections of liver show peri-vascular deposits of myeloid cells and heavy infiltration of the tissue. The spleen shows loss of normal architecture, with myeloid cells in abundance. Fluorescence activated cell sorter analysis of surface phenotype is shown in C. Spleen (spl) and bone marrow (BM) cells were prepared from Mll-loxP; Enl-loxP; Cre or from Mll-loxP; Enl-loxP mice and stained with fluorescent antibodies as indicated. Antibodies used were FITC-Gr-1 (Ly-6G) plus PE-Mac-1 (CD11b), FITC-CD8a (Ly2) or FITC-B220 (CD45R) plus PE-CD4 (L3T4). In addition, a cell line (CL) established from a Mll-loxP; Enl-loxP; Cre mouse (designated 1) was analysed with FITC-Gr-1 plus PE-Mac-1 and compared with a cell-line established from the Mll-AF9 knock-in mice (Corral et al., 1996).

FIG. 3. Reciprocal Chromosomal Translocations Occur in Mll-loxP;Enl-loxP; Cre Mice

A. DNA was prepared from tissues of mice at time of death and filter hybridisation analysis carried out using probes able to detect DNA fragments from putative chromosomal translocations. An example (specimen 1) is shown in A. Genomic DNAs from the indicated sources were digested with SphI, fragments separated on 0.8% agarose, transferred to nylon membranes and hybridised with an Mll 5′ probe (Collins et al., 2000) (left hand panel), digested with KpnI and hybridised with an Mll 3′ probe (Collins et al., 2000) (middle panel), or digested with HindIII and hybridised with an Enl 3′ probe (left hand panel), Ta=tail biopsy DNA; S=spleen DNA; L=liver DNA; K=kidney DNA; CL=an Mll-loxP; Enl-loxP; Cre cell line DNA; CCB=ES cell DNA Markers were λDNA cut with HindIII (Kb=kilobases).

B. Fluorescence in situ hybridisation of cells from bone marrow (BM) or spleen cells of Mll-loxP; Enl-loxP or Mll-loxP; Enl-loxP; Cre mice using paints for chromosome 9 (FITC) or chromosome 17 (Cy3). FISH analysis of BM from a Mll-loxP; Enl-loxP mouse or a leukaemic mouse. The FISH analysis of spleen cells from cell lines established from three independent tumours which arose in Mll-loxP; Enl-loxP; Cre mice. Representative metaphases are shown. White arrows indicate the translocated chromosomes.

FIG. 4 Chromosomal Translocations can be Detected in Young Asymptomatic Mice

A 12 day old litter from a cross between Mll-loxP; Enl-loxP homozygous mice and heterozygous Lmo2-Cre mice were used as a source of cells to examine the occurrence of chromosomal translocations. Pup 2, 7 and 10 were analysed in detail with fluorescence in situ hybridisation and genomic PCR to detect translocation products.

A. Bone marrow cells were maintained in temporary culture and metaphase spreads prepared for FISH with chromosome 9 and 17 paints. Metaphases from pups 2, 7 and 10 were analysed and representative hybridisations are shown. Mll-loxP; Enl-loxP; Cre pup 7 showed metaphases with reciprocal chromosomal translocations (pup 7, left hand panel, representative of 90% of the spreads) or normal metaphases (pup 7, middle panel, 10% of spreads). Mll-loxP; Enl-loxP; Cre pup 10 showed only normal metaphases (right hand panel). White arrows mark the position of the translocated chromosomes from pup 7.

B. Genomic DNA was isolated from BM and spleen from pups 2, 7 and 10 and PCR carried out for 30 cycles with Lmo2 specific primers (left hand panel), with Mll and Enl genomic primers (MG1+EG1) for 30 cycles (middle panel) or nested Mll and Enl genomic primers (MN+EN) for 30 cycles (right hand panel) to give total of 60 cycles. Pup 2 was a Cre negative (Mll-loxP; Enl-loxP) while pups 7 and 10 were Mll-loxP; Enl-loxP; Cre mice.

—=no template control; BM=bone marrow; Spl=spleen; T=tumour from an Mll-loxP; Enl-loxP; Cre mouse; arrows indicate the PCR band corresponding to the Lmo2 genomic product or the translocation junction product. White arrows indicate the translocated chromosomes.

C. Sequence of the BM chromosomal translocation product from a tumour.

FIG. 5.

Shows the human MLL gene location and that it has more than 30 different chromosomal translocations associated with in human leukaemias. These are mainly acute myeloid leuks (AML)

FIG. 6.

Diagram to show strategy which we now show both Mll-Enl and Mll-Af9.

FIG. 7.

The Study of leukaemia development in Mll-Enl and Mll-Af9 mice with de novo translocations induced by Cre expressed under the control of the Lmo2 promoter (i.e. in haematopoietic stem cells) or Lck-Cre (in T yymphocytes).

FIG. 8.

Leukaemia incidence curves for the relevant mice.

FIG. 9.

Any Mll chromosomal translocation may be made with this model.

FIG. 10.

Other human chromosomal translocations can be modelled with the invention described herein.

FIG. 11.

Mouse models are useful for pre-clinical testing of drugs prior to use in patients.

### DETAILED DESCRIPTION OF THE INVENTION

General Techniques

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods. In addition Harlow & Lane., A Laboratory Manual Cold Spring Harbor, N.Y, is referred to for standard Immunological Techniques.

Definitions

In general, an animal model is one in which pathological processes (for example, cancer) can be investigated, and in which the phenomenon in one or more respects resembles the same phenomenon in humans or other species of animals. An animal model, as referred to herein, is a model based on a whole organism or part thereof such as a tissue which possesses cellular organisation which replicates that seen in the whole organism. Thus, “animal model” does not include cell-based assays performed on single cells or cultures of cells. Preferably, “animal model” refers to complete animals. The animals are non-human animals, and may be mammals, birds, repltiles, fish etc. Mammals are preferred. Rodent models are convenient and within the scope of the present invention; mice and rats are preferred rodent models. Other models include canine, feline, monkey and primate models.

A “chromosomal rearrangement” is a change to the structure of a chromosome which leads to a genetic change in the subject. Rearrangements include deletions, where DNA is lost from the chromosome, insertions, inversions, duplications and translocations. Deletions can be located within a chromosome (interstitial) or can remove the end of a chromosome (terminal). Deletions can be small (intragenic), affecting only one gene, or can span multiple genes (multigenic). A deletion is a rearrangement that results in an increase in copy number of a particular chromosomal region. In tandem duplications, the duplicated regions lie right next to one another, either in the same order or in reverse order. In non-tandem duplications, the repeated regions lie far apart on the same chromosome or on different chromosomes. An inversion is a rearrangement in which a chromosomal segment is rotated 180 degrees. Inversions in which the rotated segment includes the centromere are called pericentric inversions; those in which the rotated segment is located completely on one chromosomal arm and do not include the centromere are called paracentric inversions. A translocation is a chromosomal rearrangement in which part of one chromosome becomes attached to a non-homologous chromosome (non-reciprocal), or in which parts of two non-homologous chromosomes exchange places (reciprocal).

A “site-specific recombinase” is an enzyme that catalyses site-specific recombination of nucleic acid between paired sets of cognate recognition sequence sites. Examples are set forth above. Cre Recombinase is a Type I topoisomerase from bacteriophage P1 that catalyses the site-specific recombination of DNA between loxP sites. Recombination products depend on the location and relative orientation of the lox (or loxP) sites. Two DNA species containing single lox sites will be fused. DNA between directly repeated lox sites will be excised in circular form while DNA between opposing lox sites will be inverted with respect to external sequences. Members of the Cre family cleave DNA substrates by a series of staggered cuts, during which the protein becomes covalently linked to the DNA through a catalytic tyrosine residue at the carboxy end of the recombinase motif.

A “cell type-specific promoter” is a promoter which directs expression of a nucleic acid in a cell type-specific manner; i.e. such that the said expression essentially occurs in certain tissues only. Many cell type-specific promoters are known in the art, and haematopoietic cell type-specific promoters are also known. The lmo2 promoter is preferred in the present invention. Cell type-specific promoters may “leak”, that is give rise to gene expression in cells other than the target cell type. This is acceptable, as long as sufficient levels of recombinase are achieved in the desired cell type to direct the rearrangement event therein to a sufficient level for the biological effect of the rearrangement to manifest itself.

Sites recognised by a recombinase are the cognate recognition sites. For example, Cre recognises lox or loxP sites. The loxP recognition element is a 34 bp sequence comprised of two 13 bp inverted repeats flanking an 8 bp spacer region which confers directionality. Other recognition sites, such as frt and Rs sites, are known in the art.

A “tumour”, or neoplasm, is a growth of cells resulting from abnormal regulation of cell growth leading to excessive mitotic division. Tumours may be benign, that is slow-growing and usually harmless, or malignant, that is fast growing and with a tendency to metastasise to other tissues. A “tumourigenic” event is an event that leads to or enhances tumour formation or growth.

A leukaemia, broadly speaking, is a malignant proliferation of haematopoietic cells, characterised by replacement of bone marrow by neoplastic cells. The leukaemic cells usually are present in peripheral blood, and may infiltrate other organs of the reticuloendothelial system, such as liver, spleen and lymph nodes. Leukaemia is broadly classified into acute and chronic leukaemia, with multiple distinct clinicopathologic entities subclassified in each category. Leukaemias are divided into four main types: acute myelogenous (or myeloid) leukaemia (AML), chronic myelogenous (CML), acute lymphocytic (ALL), and chronic lymphocytic (CLL).

Generation of transgenic animals

Methods for the generation of transgenic animals are generally known to those skilled in the art and are discussed in detail in First N, Haseltine FP., Transgenic animals: Stoneham, UK: Butterworth-Heinmann, 1991; and Grosveld F, Kollias G., Transgenic animals: San Diego, Calif.: Academic Press, 1992. Commonly used methods include the ES cell method and pronuclear microinjection. In the context of the present invention, ES cell techniques are preferred.

In this technique, embryonic stem cells (ES cells) are harvested from the inner cell mass (ICM) of animal blastocysts. They can be grown in culture and retain their full potential to produce all the cells of the mature animal, including its gametes. The cultured cells are exposed to the DNA which is intended to be inserted into the cells so that some cells will incorporate it. The cells are then tested for successful incorporation of the DNA, usually by selecting for the presence of a neo marker, and injected into the inner cell mass of an animal blastocyst. A proportion of the cells of the embryo resulting from the blastocyst will incorporate the desired DNA.

A pseudopregnant animal is prepared and the embryos transferred into the uterus. A proportion of the embryos will develop into animals carrying the inserted DNA.

Targeted gene insertion requires some additional steps. The vector used to transfer the DNA typically includes:
the desired gene
neo^{r}, a gene that encodes an enzyme that inactivates the antibiotic neomycin (and its relatives)
tk, a gene that encodes thymidine kinase, an enzyme that phosphorylates the nucleoside analogue gancyclovir. DNA polymerase fails to discriminate against the resulting nucleotide and inserts this non-functional nucleotide into freshly-replicating DNA.

The culture of ES cells is treated with a preparation of vector DNA:
Most cells fail to take up the vector; these cells will be killed if exposed to neomycin.
In a few cells: the vector is inserted randomly in the genome. In random insertion, the entire vector, including the tk gene, is inserted into host DNA. These cells are resistant to neomycin but killed by gancyclovir.
In still fewer cells: homologous recombination occurs. Stretches of DNA sequence in the vector find the homologous sequences in the host genome and the region between these homologous sequences replaces the equivalent region in the host DNA.

Positive selection of transformed cells is achieved by culturing all cells in neomycin. The cells (the majority) that fail to take up the vector are killed. Negative selection of all cells (tk⁺) in which the vector was inserted randomly by culturing cells surviving the neomycin selection is performed by culturing the cells in gancyclovir. This step leaves a population of cells transformed by homologous recombination (enriched several thousand fold). These cells are injected into the inner cell mass of animal blastocysts.

Methods for introduction of recombinase specific sites into the genome of a transgenic animal are described in Collins et al., (2000) EMBO Reports 1:127-132.

The insertion of genes encoding a site-specific recombinase under the control of a cell type-specific promoter has been described in the art. For example, Cre recombinase has been placed under the control of promoters for:

| | |
|---|---|
| αA-crystallin | Eye lens [Lasko et al., (1992) PNAS 89: 6232-6236] |
| Calcium/calmodulin-dependent | Forebrain (CA1 pyramidal cells) [Tsien et al., (1996) |
| protein kinase IIα | Cell 87: 1317-1326] |
| P0 gene | Schwann cells [Akagi et al., (1997) NAR 25: 1766-1773] |
| Pro-opiomelanocortin | Pituitary gland (intermediate lobe) [Akagi et al., (1997) |
| | NAR 25: 1766-1773] |
| Interphotoreceptor retinoid | Retina (photoreceptor cells) [Akagi et al., (1997) NAR |
| binding protein | 25: 1766-1773] |
| wnt-1 | Nervous system [Danielian et al., (1998) Curr. Biol. |
| | 8: 1323-1326] |
| Engrailed-2 | Nervous system [Zinyk et al. (1998) Curr Biol 8: 665-668] |
| Proximal lck | T cells [Vooijs et al., (1998) Oncogene 17: 1-12] |
| CD19 (knock in) | B cells [Rickert et al., (1997) NAR 25: 1317-1318] |
| α myosin heavy chain | Heart (ventricular myocytes) [Agah et al., (1997) J Clin |
| | Invest 100: 169-179] |
| Myosin light chain 2v (knock | Heart (ventricular myocytes) [Chen et al., (1998) |
| in) | Development 25: 1943-1949; Chen et al., (1998) J Biol |
| | Chem 273: 1252-1256] |
| Muscle creatine kinase | Skeletal muscle, heart [Bruening et al., (1998) Mol Cell |
| | 2: 559-569; Wang et al., (1999) Nat Genet 21: 133-137] |
| Insulin | Pancreas (β cells) [Ray et al., (1998 BBRC 253: 65-69; |
| | Postic et al., (1999) J Biol Chem 274: 305-315] |
| Albumin enhancer/promoter | Liver [Postic et al., (1999) J Biol Chem 274: 305-315] |
| Whey acidic protein | Mammary gland, brain [Wagner et al., (1997) NAR |
| | 25: 4323-4330] |
| β-lactoglobulin | Mammary gland [Selbert et al., (1998) Transgenic Res |
| | 7: 387-396] |
| Adipose protein 2 | Adipose tissue [Barlow et al., (1997) NAR 25: 2543-2545] |
| Keratin 5 | Skin (basal keratinocytes) [Tarutani et al., (1997) |
| | PNAS 94: 7400-7405] |

The gene encoding the recombinase may be inserted in the same procedure as that used to insert the cognate sites, or by a separate procedure. It can be introduced into the same animal, or a separate animal. For example, a transgenic animal carrying the recombinase gene, introduced by ES cell transformation or pronuclear microinjection, can be crossed with an animal carrying the cognate sites to produce an animal of the desired genotype.

Site-Specific Recombination

Site-specific recombinase enzymes include the Int recombinase of bacteriophage λ (with or without Xis) (Weisberg, R. et al., in Lambda II, (Hendrix, R., et al., Eds.), Cold Spring Harbor Press, Cold Spring Harbor, N.Y., pp. 211-50 (1983); TpnI and the β-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpoIVC recombinase (Sato, et al., J. Bacteriol. 172:1092-98 (1990)); the Flp recombinase (Schwartz & Sadowski, J. Molec. Biol., 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)). Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al.,(Mol Cell. Biochem., 92:107-16 (1990)); and Cregg & Madden (Mol. Gen. Genet., 219:320-23 (1989)).

Cre recombinase has been purified to homogeneity, and its reaction with the loxP site has been extensively characterised (Abremski & Hess J. Mol. Biol. 259:1509-14 (1984); Gopaul and van Duyne, (1999) Curr Opin Struct Biol 9:14-20). Cre protein has a molecular weight of 35,000 and can be obtained commercially from New England Nuclear/DuPont. The cre gene (which encodes the Cre protein) has been cloned and expressed (Abremski, et al., Cell 32:1301-11 (1983)). The Cre protein mediates recombination between two loxP sequences (Sternberg, et al., Cold Spring Harbor Symp. Quant. Biol. 45:297-309 (1981)), which may be present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al., Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Modelling of Chromosomal Rearrangements

Chromosomal rearrangements are commonplace in cancers and are efficiently modelled by the method of the invention. The use of a recombinase-catalysed rearrangement at recombinase-specific sites permits the generation of realistic models of chromosomal rearrangements.

Many chromosomal rearrangements are known in the art, which can be modelled in accordance with the present invention. These include:

1. Chronic Myeloproliferative Diseases (MPD)
Agnogenic myeloid metaplasia
Atypical Chronic Myeloid Leukaemia (aCML)
Chronic Myelomonocytic Leukaemia (CMML)
Chronic myelogenous leukaemia (CML)
Essential thrombocythemia
Idiopathic myelofibrosis
Idiopathic thrombocythemia
Juvenile Chronic Myelogenous Leukaemia (JCML)
Polycytemia vera
del(3q) in myeloid malignancies
del(16)(q22)
del(20q) in myeloid malignancies
del(5q) in myeloid malignancies
del(9q) solely
i(17q) in myeloid malignancies
idic(X)(q13)
ins(3;3)(q26;q21q26)
inv(16)(p13q22)
inv(3)(q21q26)
t(12;13)(p12;q12-14)
t(12;22)(p13;q11-12)
t(16;16)(p13;q22)
t(1;18)(q10;q10)
t(1;3)(p36;p21)
t(1;3)(p36;q21)
t(1;7)(q10;p10)
t(3;3)(q21;q26)
t(5;10)(q33;q21)
t(5;11)(q31;q23)
t(5;12)(q33;p13)
t(7;11)(p5;p15)
t(8;14)(q11;q32)
t(8;22)(p11;q11)
t(9;12)(p24;p13)
t(9;12)(q34;p13)
t(9;22)(q34;q11) in CML
t(Y;1)(q12;q12)

2. Myelodysplastic Syndromes (MDS)
11q23 rearrangements in leukaemia
12p abnormalities in myeloid malignancies
Childhood myelodysplastic syndromes
Chronic Myelomonocytic Leukaemia (CMML)
Congenital leukaemias
Infant leukaemias
Neonatal leukaemias
del(13q) in myeloid malignancies
del(16)(q22)
del(17p) in myeloid malignancies
del(20q) in myeloid malignancies
del(5q) in myeloid malignancies
del(9q) solely
i(17q) in myeloid malignancies
idic(X)(q13)
ins(3;3)(q26;q21q26)
inv(16)(p13q22)
inv(3)(q21q26)
t(11;16)(q23;p13)
t(11;17)(q23;q25)
t(12;13)(p12;q12-14)
t(12;22)(p13;q11-12)
t(16;16)(p13;q22)
t(16;21)(q24;q22)
t(1;16)(q11;q11)
t(1;18)(q10;q10)
t(1;19)(p13;p13.1)
t(1;3)(p36;p21)
t(1;3)(p36;q21)
t(1;7)(q10;p10)
t(2;11)(p21;q23)
t(3;12)(q26;p13)
t(3;21)(q26;q22)
t(3;3)(q21 ;q26)
t(5;11)(q31;q23)
t(5;12)(q33;p13)
t(5;7)(q33;q11)
t(6;8)(q27;p12)
t(6;9)(p23;q34)
t(7;12)(q36;p13)
t(8;13)(p12;q12)
t(9;12)(q22;p12)
t(Y;1)(q12;q12)
3. Treatment Related Leukaemias (t-ANLL)
11q23 rearrangements in leukaemia
11q23 rearrangements in therapy related leukaemias
12p abnormalities in myeloid malignancies
Biphenotypic Acute Leukaemia (BAL)
Childhood myelodysplastic syndromes
del(16)(q22)
del(17p) in myeloid malignancies
del(5q) in myeloid malignancies
inv(16)(p13q22)
t(10;11)(p12;q23)
t(11;12)(p15;q13)
t(11;15)(q23;q14)
t(11;16)(q23;p13)
t(11;17)(q23;p13)
t(11;17)(q23;q25)
t(11;19)(q23;p13.1)
t(11;19)(q23;p13.3)
t(11;22)(q23;q13)
t(15;17)(q22;q21)
t(16;16)(p13;q22)
t(16;21)(q24;q22)
t(17;21)(q11.2;q22)
t(18;21)(q21;q22)
t(19;21)(q13.4;q22)
t(1;21)(p36;q22)
t(1;3)(p36;p21)
t(1;3)(p36;q21)
t(1;7)(p36;q34)
t(1;7)(q10;p10)
t(3;11)(p21;q23)
t(3;11)(q25;q23)
t(3;11)(q28;q23)
t(3;21)(q26;q22)
t(4;11)(q21;q23)
t(4;12)(q11-q21;p13)
t(5;11)(q31;q23)
t(6;11)(q27;q23)
t(6;9)(p23;q34)
t(8;16)(p11;p13)
t(8;21)(q22;q22)
t(9;11)(p22;q23) 4. Acute Non Lymphocytic Leukaemias (ANLL)
11q23 rearrangements in leukaemia
12p abnormalities in myeloid malignancies
Acute Erythroid leukaemias
Acute basophilic leukaemia
Biphenotypic Acute Leukaemia (BAL)
Congenital leukaemias
Infant leukaemias
M0 acute non lymphocytic leukaemia (M0-ANLL)
Neonatal leukaemias
Systemic mast cell disease (SMCD)
del(13q) in myeloid malignancies
del(16)(q22)
del(17p) in myeloid malignancies
del(20q) in myeloid malignancies
del(5q) in myeloid malignancies
del(9q) solely
idic(X)(q13)
ins(3;3)(q26;q21q26)
inv(14)(q11;q32.1)
inv(16)(p13q22)
inv(3)(q21q26)
t(10;11)(p11.2;q23)
t(10;11)(p12;q23)
t(10;11)(p13;q21)
t(11;16)(q23;p13)
t(11;17)(q13;q21)
t(11;17)(q23;p13)
t(11;17)(q23;q12)
t(11;17)(q23;q21)
t(11;17)(q23;q25)
t(11;19)(q23;p13.1)
t(11;19)(q23;p13.3)
t(11;22)(q23;q11.2)
t(12;13)(p12;q12-14)
t(12;22)(p13;q11-12)
t(14;14)(q11;q32.1)
t(15;17)(q22;q21)
t(16;16)(p13;q22)
t(16;21)(p11;q22)
t(16;21)(q24;q22)
t(17;21)(q11.2;q22)
t(1;11)(q21;q23)
t(1;12)(q25;p13)
t(1:18)(q25;q23)
t(1;19)(p13;p13.1)
t(1;19)(q23;p13)
t(1;22)(p13;q13)
t(1;3)(p36;p21)
t(1;3)(p36;q21)
t(1;7)(p36;q34)
t(1;7)(q10;p10)
t(2;11)(p21;q23)
t(3;12)(q26;p13)
t(3;21)(q26;q22)
t(3;3)(q21;q26)
t(3;5)(q25;q34)
t(4;11)(q21;q23)
t(4;12)(q11-q21;p13)
t(5;11)(q31;q23)
t(5;11)(q35;p15.5)
t(5;14)(q33;q32)
t(5;17)(q35;q21)
t(6;11)(q27;q23)
t(6;8)(q27;p12)
t(6;9)(p23;q34)
t(7;11)(p15;p15)
t(7;12)(q36;p13)
t(8;16)(p11;p13)
t(8;21)(q22;q22)
t(9;11)(p22;q23)
t(9;11)(q34;q23)
t(9;12)(q34;p13)
t(9;22)(q34;q11) in ANLL
t(X;11)(q13;q23)
t(X;6)(p11;q23)

5. B-Cell Acute Lymphocytic Leukaemias (13-ALL)
11q23 rearrangements in leukaemia
12p rearrangements in ALL
9p Rearrangements in ALL
Biphenotypic Acute Leukaemia (BAL)
Congenital leukaemias
High hyperdiploid acute lymphoblastic leukaemia
Infant leukaemias
Near haploid acute lymphoblastic leukaemia
Neonatal leukaemias
Severe hypodiploid acute lymphoblastic leukaemia
del(6q) abnormalities in lymphoid malignancies
dic(9;12)(p11-13;p11-12)
dic(9;20)(p11-13;q11)
ins(5;11)(q31;q13q23)
inv(14)(q11;q32.1)
t(10;11)(p12;q23)
t(10;11)(p13;q21)
t(11;17)(q23;p13)
t(11;17)(q23;q25)
t(11;19)(q23;p13.3)
t(12;13)(p12;q12-14)
t(12;21)(p12;q22)
t(14;14)(q11;q32.1)
t(14;18)(q32;q21)
t(17;19)(q22;p13)
t(18;22)(q21;q11)
t(1;18)(q25;q23)
t(1;19)(q23;p13)
t(1;3)(p36;p21)
t(2;11)(p21;q23)
t(2;14)(p13;q32)
t(2;18)(p11;q21)
t(2;8)(p12;q24)
t(4;11)(q21;q23)
t(4;12)(q11-q21;p13)
t(5;11)(q31;q23)
t(5;14)(q31;q32)
t(5;15)(p15;q11-13)
t(8;14)(q24;q11)
t(8;14)(q11;q32)
t(8;14)(q24;q32)
t(8;22)(q24;q11)
t(9;12)(p24;p13)
t(9;12)(q34;p13)
t(9;22)(q34;q11) in ALL

6. T-Cell Acute Lymphocytic Leukaemias (T-ALL)
11q23 rearrangements in leukaemia
12p rearrangements in ALL
1p32 rearrangements
9p Rearrangements in ALL
Biphenotypic Acute Leukaemia (BAL)
del(6q) abnormalities in lymphoid malignancies
del(9q) solely
dic(9;12)(p11-13;p11-12)
inv(14)(q11;q32.1)
t(10;11)(p13;q21)
t(10;14)(q24;q11)
t(11;14)(p13;q11)
t(11;14)(p15;q11)
t(11;19)(q23;p13.3)
t(12;13)(p12;q12-14)
t(14;14)(q11;q32.1)
t(1;14)(p32;q11)
t(1;19)(q23;p13)
t(1;3)(p32;p21)
t(1;5)(p32;q31)
t(1;7)(p32;q34)
t(2;14)(p13;q32)
t(4;11)(q21;p15)
t(4;11)(q21;q23)
t(5;14)(q35;q32)
t(5;17)(q13;q21)
t(6;11)(q27;q23)
t(7;10)(q34;q24)
t(7;11)(q35;p13)
t(7;9)(q34;q32)
t(8;14)(q24;q11)
t(9;12)(p24;p13)
t(9;22)(q34;q11) in ALL
t(X;11)(q13;q23)

7. Non Hodgkin Lymphomas (NHL)
3q27 rearrangements in non Hodgkin lymphoma
Anaplasic large cell lymphoma (ALCL)
Angioimmunoblastic T-cell lymphoma
Burkitt's lymphoma (BL)
Classification of T-Cell disorders
Diffuse large cell lymphoma
Follicular lymphoma (FL)
Hodgkin's disease
Lymphoepithelioid lymphoma
Lymphoplasmacytic lymphoma
Mantle cell lymphoma (incomplete)
Marginal Zone B-cell lymphoma
Small lymphocytic lymphoma
T-cell large granular lymphocyte leukaemia
Waldenstrom's macroglobulinemia (WM)
del(11q) in non-Hodgkin's lymphoma (NHL)
del(13q) in non-Hodgkin's lymphoma
del(17p) in non-Hodgkin's lymphoma (NHL)
del(6q) abnormalities in lymphoid malignancies
del(7q) in non-Hodgkin's lymphoma (NHL)
dic(9;12)(p11-13;p11-12)
inv(14)(q11;q32.1)
inv(2)(p23q35)
t(10;14)(q24;q11)
t(11;14)(q13;q32)
t(11;18)(q21;q21)
t(14;14)(q11;q32.1)
t(14;18)(q32;q21)
t(14;19)(q32;q13)
t(18;22)(q21;q11)
t(1;13)(q32;q14)
t(1;14)(p22;q32) in non Hodgkin's lymphoma (NHL)
t(1;16)(q11;q11)
t(1;19)(q23;p13)
t(1;2)(q25;p23)
t(1;3)(p36;p21)
t(1;7)(q21;q22)
t(2;14)(p13;q32)
t(2;18)(p11;q21)
t(2;22)(p23;q11.2)
t(2;3)(p12;q27)
t(2;3)(p23;q21)
t(2;5)(p23;q35)
t(2;8)(p12;q24)
t(3;13)(q27;q14)
t(3;14)(q21;q32)
t(3;14)(q27;q32)
t(3;22)(q27;q11)
t(3;4)(q27;p13)
t(3;Var)(q27;Var) in non Hodgkin lymphoma
t(6;8)(q11;q11)
t(6;8)(q27;p12)
t(7;10)(q34;q24)
t(8;13)(p12;q12)
t(8;14)(q24;q11)
t(8;14)(q24;q32)
t(8;22)(q24;q11)
t(9;14)(p13;q32)
t(X;2)(q11;p23)

8. Chronic Lymphoproliferative Diseases (CLD)
1q rearrangements in multiple myeloma
B-cell prolymphocytic leukaemia (B-PLL)
Chronic lymphocytic leukaemia (CLL)
Fibrogenesis imperfecta ossium
Hairy Cell Leukaemia (HCL) and Hairy Cell Leukaemia Variant (HCL-V)
Multiple myeloma
Plasma cell leukaemia (PCL)
Splenic lymphoma with villous lymphocytes
T-cell prolymphocytic leukaemia (T-PLL)
del(13q) in chronic lymphoproliferative diseases
del(6q) in Multiple Myeloma
del(13q) in multiple myeloma
del(6q) abnormalities in lymphoid malignancies
inv(14)(q11;q32.1)
t(11;14)(p11;q32)
t(11;14)(q13;q32)
t(11;14)(q13;q32) in multiple myeloma
t(14;14)(q11;q32.1)
t(14;18)(q32;q21)
t(14;19)(q32;q13)
t(18;22)(q21;q11)
t(1;3)(p36;q21)
t(2;14)(p13;q32)
t(2;18)(p11;q21)
t(4;14)(p16;q32)
t(9;14)(p13;q32)

See also Mitelman's Catalog of Chromosome Aberrations in Cancer '98, John Wiley & Sons, N.Y., USA; ISBN 0-471-17603-6.

Advantageously, the disease modelled by the methods of the invention is leukaemia. Preferably, it is a myeloid or lymphoid leukaemia, particularly leukaemia associated with human t(11;19), equivalent to t(9;17) in mouse, to produce an Mll-Enl fusion.

Sequence from the rearrangement sites is obtained from publicly-available sources, such as GenBank, and used to construct homologous recombination vectors which insert recombinase specific sites, such as lox sites, into the regions of interest. The sites can be configured to cause a deletion (e.g. using a “floxed” (loxP-flanked) section of DNA), inversion (using, for example, two loxP sites inserted in opposing orientations), translocation or other rearrangement of the chromosomal locus.

Disease models of chromosomal translocations according to the invention replicate the aetiology of human chromosomal translocations with surprising fidelity. Preferably, the translocation according to the inventions results in an Mll-Enl fusion, which is associated with leukaemia in humans. As in humans, the chromosomal translocation between mouse Mll and Enl results in leukaemia. The data from our study shows that mice with the compound genotype comprising loxP recombination sites with Mll and Enl genes develop leukaemia only if Cre recombinase is expressed. The Mll-Enl chromosomal translocation thus always results in malignancy. The onset of malignancy is rapid which is consistent with retroviral transduction studies (Ayton and Cleary, 2001; Lavau et al., 1997; Slany et al., 1998) but in the translocator mice, the development of disease occurs after a somatic event (i.e. chromosomal translocation) and is not due to the simultaneous transplantation of many transduced, Mll-Enl expressing cells. This implies that the effect of Mll-Enl is paramount in the leukaemic process, perhaps the only event needed for leukaemia. It is significant that young mice with low number of cells with chromosomal translocations (i.e. only detectable by nested PCR) co-exist with littermates with high numbers of chromosomal translocation-positive cells. This must reflect the time at which the inter-chromosomal event occurred (which cannot be defined in individual mice nor how long after overt disease takes to manifest itself) and the putative selectivity which is conferred on the cell which undergoes chromosomal translocation. The early appearance of large proportions of translocation carrying cells (i.e. as early as 12 days) further suggests that the emergence of overt haematopoietic malignancy is wholly dependent on the Mll-Enl fusion gene, without the intervention of further mutational events.

The Lmo2 gene is needed for the development of adult haematopoiesis (Yamada et al., 1998) and Cre-expression from the Lmo2-Cre knock-in gene occurs in bone marrow. The major site of the chromosomal translocation is therefore likely to be bone marrow cells which is supported by the PCR and FISH analysis of 12 day old mice (FIG. 4) where concentrations of cells with chromosomal translocations are high in bone marrow but not in spleen. The leukaemia in this model most likely initiates in bone marrow progenitors, which progress and migrate to remote locations such as spleen. At the time of overt disease, the abnormal myeloid cells are found to be invading somatic tissues with typical, large peri-vascular deposits in liver and kidney.

Detection of Tumour Formation

The presence of tumours in animals according to the invention is possible by a variety of methods. These include genetic testing, testing for tumour markers, physical observation, cytological assays and the like.

Tumour markers are substances that can be detected in higher than normal amounts in the blood, urine, or body tissues of animals with certain types of cancer. A tumour marker may be produced by the tumour itself, or by the body in response to a cancer presence. When diagnosing cancer, blood and tumour tissue biopsies may be tested. For example, the following markers are known to associated with tumours:

| | Primary |
|---|---|
| Tumour Marker | Cancer Site |
| Antidiuretic Hormone (ADH) | Small cell lung cancer, |
| | adenocarcinoma |
| Alpha-feto protein (AFP) | Liver, germ cell cancer of ovaries |
| | or testis |
| BTA (Bladder Tumour Antigen) | Bladder |
| CA15-3 (carbohydrate antigen 15-3) | Breast |
| CA19-9 | Pancreas, colorectal |
| CA125 | Ovarian |
| Calcitonin | Thyroid medullary carcinoma |
| Carcinoembryonic antigen (CEA) | Colon, Lung |
| Creatin-kinase-BB | Breast, ovary, colon, prostate |
| hCG (human chorionic gonadotropin) | Trophoblastic disease |
| Lactic dehydrogenase (LDH) | Lymphoma, seminoma, acute |
| | leukaemia, metastatic carcinoma |
| Neuron-specific enolase (NSE) | Neuroblastoma, small cell lung |
| | cancer |
| NMP 22 | Bladder |
| Prostatic acid phosphatase (PAP) | Metastatic cancer of prostate, |
| | myeloma, lung cancer, osteogenic |
| | sarcoma |
| Prostate specific antigen (PSA) | Prostate |

Tests for tumour markers are available commercially; for example, immunoassays for particular polypeptides may be used to assess their presence and/or amounts in biological samples.

Cytological assays are frequently used in the detection of leukaemia, for example by analysis of various tissues post mortem or analysis of bone marrow tissue obtained by biopsy. Another technology useful for detecting leukaemia, multiparameter immunological detection by flow cytometry, is claimed to be capable of detecting one leukaemic cell among 10,000 normal cells. The principle behind this assay is that leukaemia cells display certain surface, cytoplasmic and nuclear leukocyte antigens which normal cells do not. A fluorescent dye is tagged to an antigen-specific monoclonal antibody, incubated with a sample of bone marrow aspirate from a leukaemic animal, and the cells monitored by flow cytometry for the attachment of fluorescent dyes to the cells. If the cells fluoresce, they possess leukaemia antigens (see Coustan-Smith et al., Lancet 1998; 351:550-4).

Uses of Transgenic Animals According to the Invention

The de novo chromosomal translocations in mice are the closest to natural, human chromosomal translocations of any model thus far. The finding that inter-chromosomal translocations between Mll and Enl in mice invariably lead to leukaemia suggests that the Cre-loxP in vivo approach (Buchholz et al., 2000; Collins et al., 2000) can be employed, with appropriately specific Cre expressing mice, to any pair of genes as long as the orientation to the centromere does not result in dicentric aberrant chromosomes. The employment of cell-specific Cre expression or inducible Cre will increase the versatility of this approach and make experiments possible which evaluate hierarchies of genetic changes associated with human cancers with chromosomal translocations. In addition, Cre-dependent, inter-chromosomal translocations result in leukaemias with reciprocal translocated chromosomes, which is a unique feature of this approach, and which may well be a crucial issue in cases where the involvement of both translocated chromosomes has been implicated (He et al., 1997).

The translocator mouse approach should not only provide the basis for generating chromosomal translocations to mimic the diversity of breakpoints in leukaemias and sarcomas but, in addition, should provide an experimental framework for exploring the role of translocations in epithelial tumours, where recurrent and non-recurrent translocations are found (Mitelman et al., 2000). While tumours of epithelial cell origin do have chromosomal translocations (Dutrillaux, 1998), they do not appear to fall into the recurrent category found in leukaemias and sarcomas. Whether these arise due to genetic instability alone or whether they have, in addition, pathogenic consequences for the individual tumours (idiopathic) is not known. The ability to make de novo translocations to recapitulate idiopathic chromosomal translocations is an invaluable tool to ascertain the significance of these aberrant chromosomes in cancer.

The invention is further described, fur the purpose of illustration, in the following examples:

### EXPERIMENTAL PROCEDURES

Gene Targeting & Production of Translocator Mice

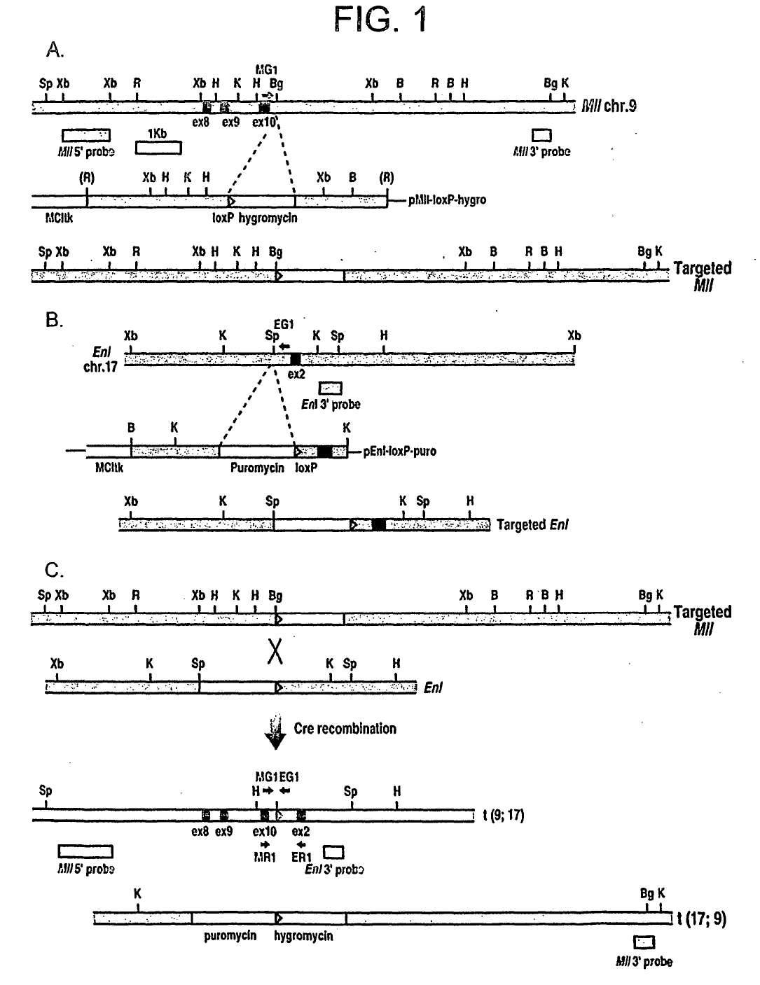

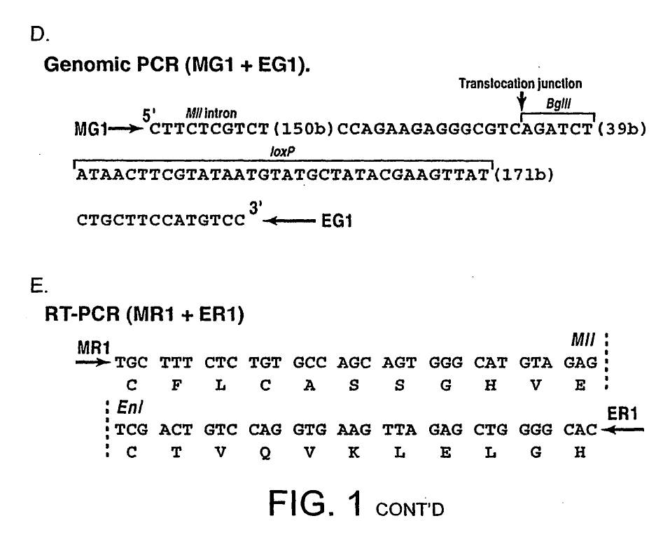

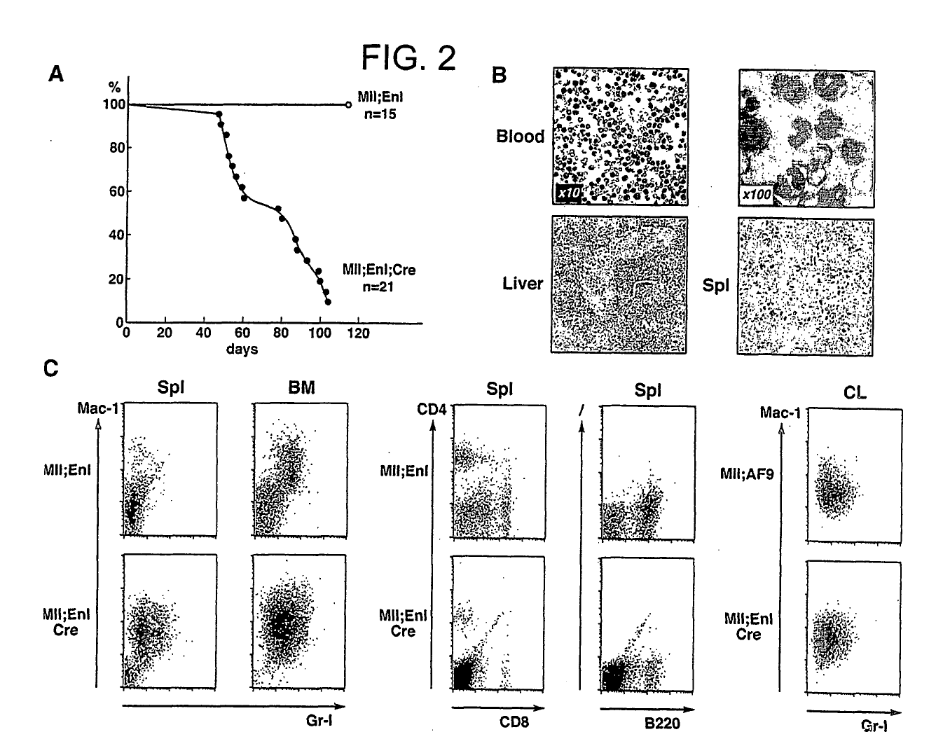

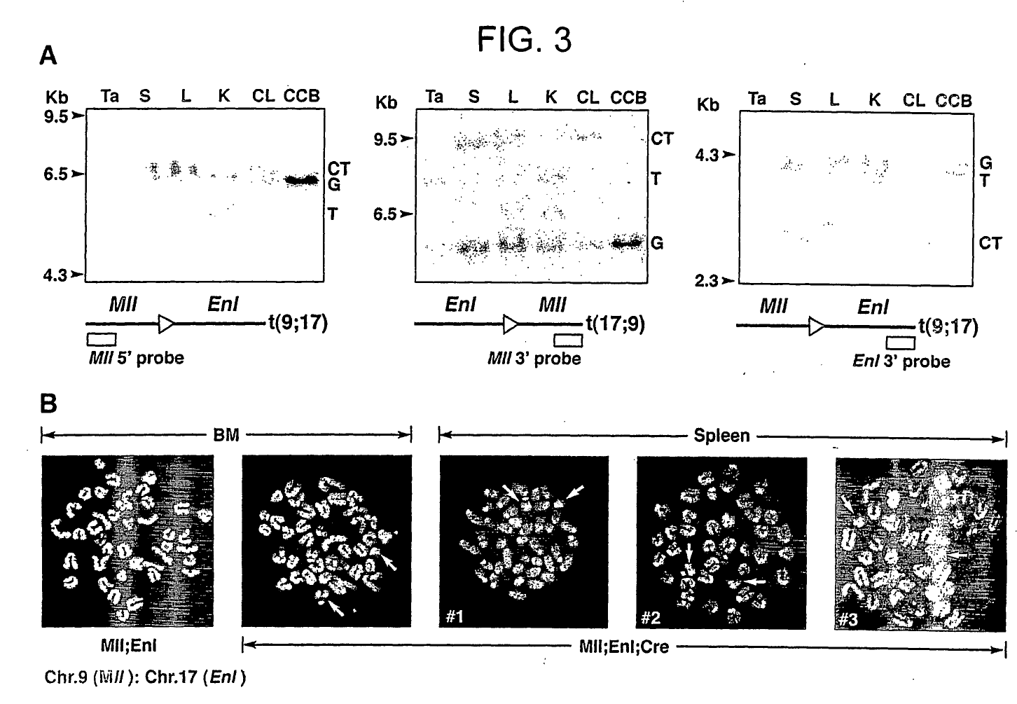

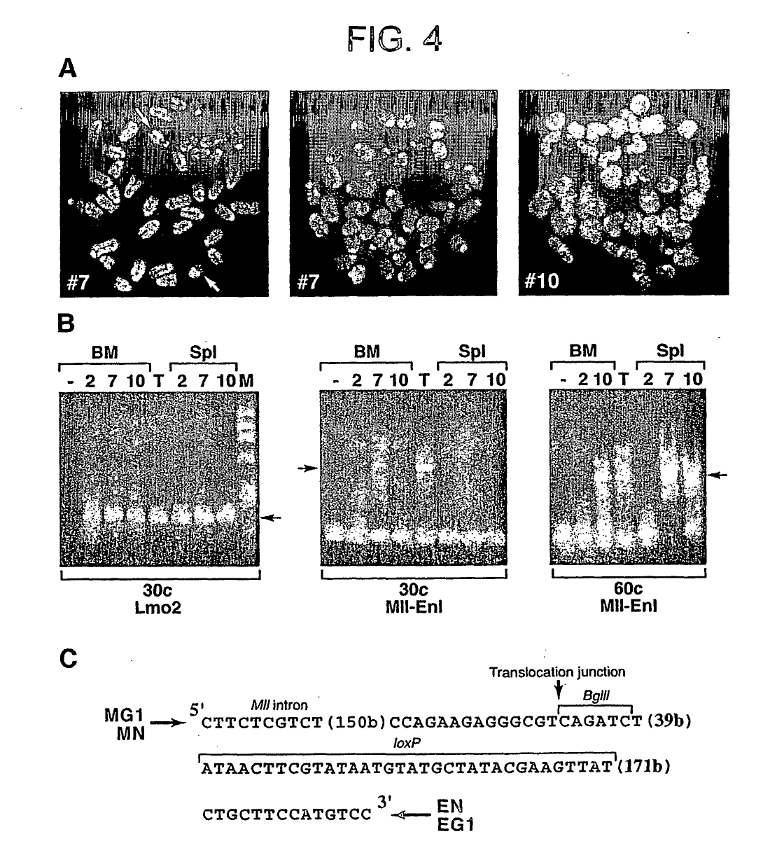

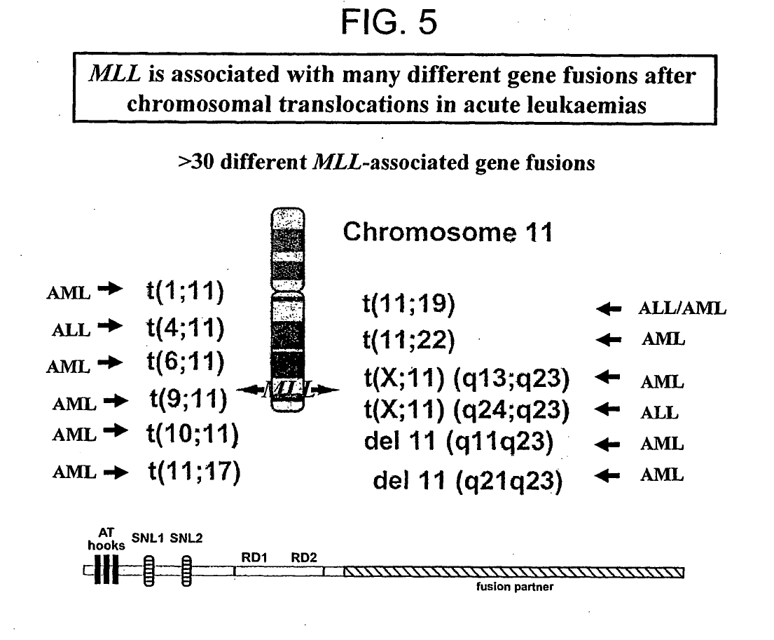

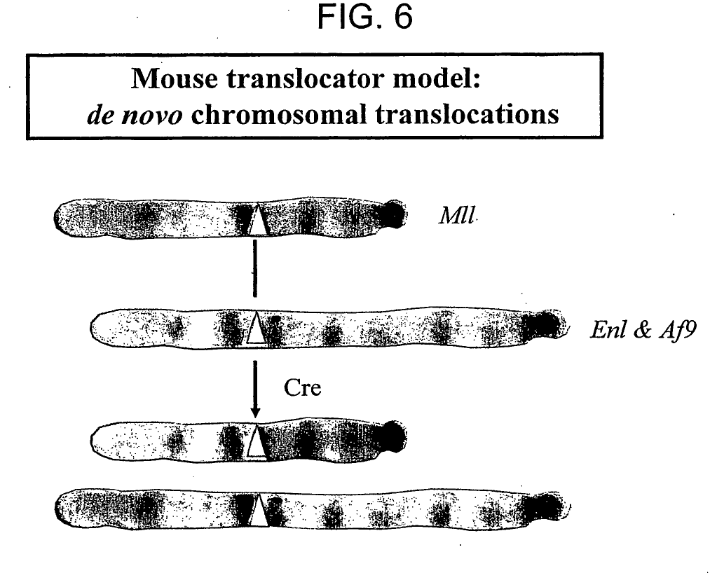

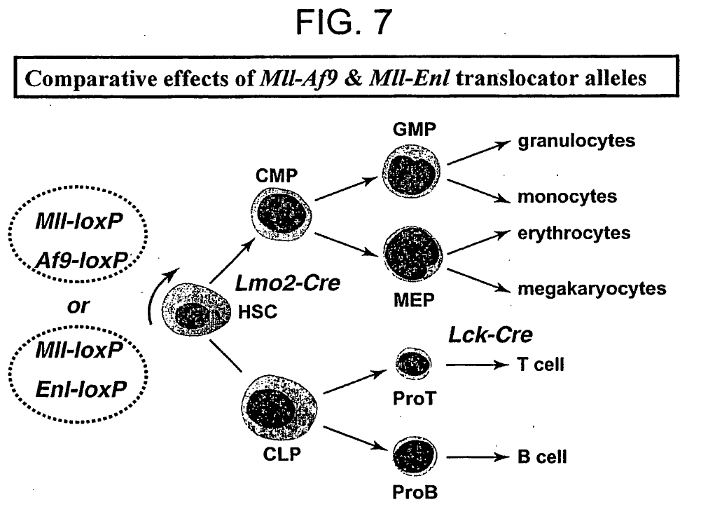

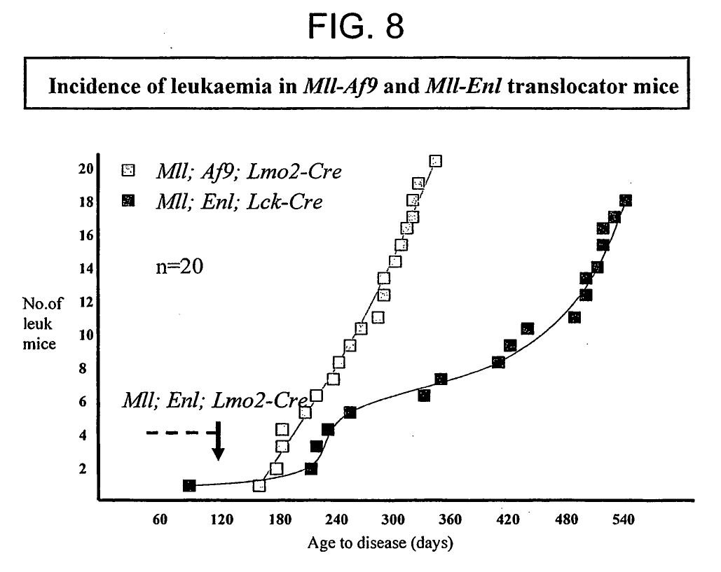

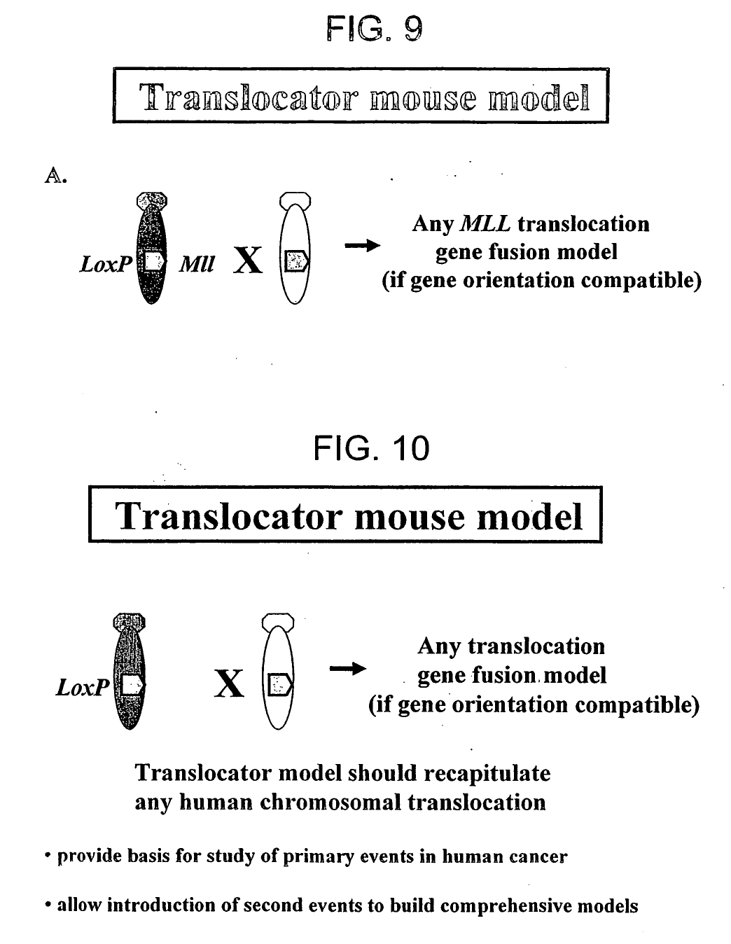

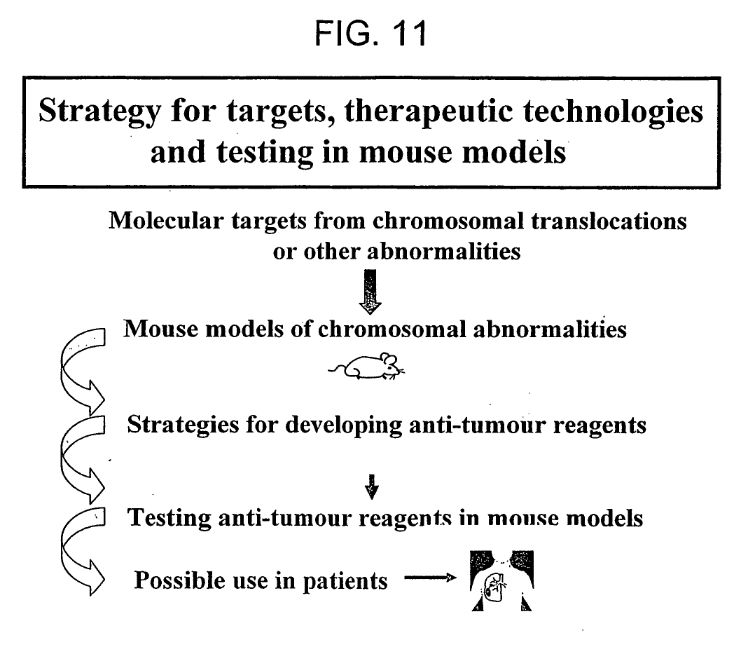

The mouse Mll genomic fragment with exon 10 and loxP has been previously described (Collins et al., 2000). Genomic λ phage DNA clones of the mouse Enl gene were isolated from a library of 129 DNA using a human cDNA clone made by PCR amplifying a 350 bp fragment including exon 2. The loxP-hygromycin and puromycin-loxP cassettes (Collins et al., 2000) were cloned into BglII (Collins et al., 2000) and SphI site of Mll and Enl respectively, to generate the two targeting vectors pMll-loxP-hygro and pEnl-loxp-puro (FIGS. 1A and B). Homologous recombination was carried out into CCB ES cells as described (Robertson, 1987) using either hygromycin or puromycin-resistant mouse embryonic fibroblast feeders as appropriate (Johnson et al., 1995; Linnell et al., 2001). Targeted ES clones were identified by filter hybridisation (LeFranc et al., 1986) using 5 and 3′ flanking probes from Mll genomic DNA (FIG. 1A). The 5′ Enl flanking probe was a 1 kb BamHI+XbaI fragment located 5′ of exon 2 and the 3′ probe was a KpnI-SphI fragment (Fig. 1B). A clone with targeted Mll was re-transfected with the pEnl-loxP-puro clone to make double targeted ES clones. To determine if inter-chromosomal translocation between Mll and Enl could occur, double targeted clones were transiently transfected with the Cre recombinase expression vector pPGKCrebpA, using electroporation (Collins et al., 2000). After 72 hours, cells were harvested, RNA was prepared using Trizol (Sigma) and DNA using a standard phenol-chloroform procedure. The presence of the Mll-Enl translocation chromosome t(9;17) was confirmed by genomic PCR using 0.5 μg DNA in a PCR reaction with 5 ng primers MG1+EG1 in the following conditions; initial 95° C. 2′, 95° C. 30 sec touchdown (Feinberg and Vogelstein, 1983) 65°-56° C., 2 cycles each for 30 sec, 10 cycles at 55° C. and extend 72° C. for 1′ with final 10′ extension. A PCR product of 470 bp was visualised on 1.3% agarose gels and cloned into pGEM-T (Promega) for sequencing (FIG. 1D). RT-PCR was carried out with cDNA synthesised using the ES cell RNA. cDNA was synthesised with an oligo-dT primer using 5 μg total RNA in final volume of 100 μl. 1 μl of cDNA was amplified in a 25 μl PCR reaction containing 5 ng of primers MR1+ER1 (FIG. 1E) in the following conditions; 0.95° C. 2′, 35 cycles of 95° C. for 30 sec, 60° C. for 1 ′, 72° C. for 1′, then 1 μl was nested for a further 30 cycles. A PCR product of 400 bp was visualised on 1.3% agarose gels and cloned into pGEM-T (Promega) for sequencing.

Phenotypic Analysis

Mice were strictly monitored and were sacrificed at the first signs of ill health. Blood smears were prepared at time of death and stained with May-Grunwald-Giemsa (MGG) stain. Full post-mortem examination was carried out and tissues fixed in 10% buffered formalin. After wax embedding, 4 μM sections were made and stained with haematoxylin and eosin. Blood films and sections were analysed and images made with a Zeiss Axioplan microscope. Fluorescence activated cell sorter analysis was carried out on a FACSCalibur and data collected using Cellquest software (BD). Antibodies used at 1/10 dilution in PBS were directly coupled to the respective fluorochrome (Pharmingen). Antibodies used were PE-labelled Mac-1 (CD11b), FITC-labelled Gr-1 (Ly-6G), FITC-labelled CD4 (L3T4), PE-labelled CD8a (Ly-2), FITC-labelled B220 (CD45R). Spleen and bone marrow cells were prepared as single cell suspensions by passing through a 70 μM cell strainer (BD), washed in cold PBS and suspended to a concentration of <5×10⁷ cells/ml in cold PBS/1%FCS. Antibody dilutions were added to 100 μl of cells and incubated on ice for 1 hour. The appropriate isotype controls were used for each antibody.

Determination of Chromosomal Translocations

Tumour cells (spleen or bone marrow) were prepared as single cell suspensions and cultured in 5% CO₂ in RPMI medium supplemented with 20% foetal calf serum, 20 u/ml IL2, 10 u/ml IL6, IL7 and GM-CSF (Roche), 5% WEHI231 confluent culture supernatant (IL3 source (Karasuyama and Melchers, 1988)) plus 100 μg/ml penicillin-streptomycin and 200 μg/ml gentamycin. For primary cultures, medium was replenished as necessary and non-adherent cells retained for further use. Fluorescence in situ hybridisation (FISH) was carried out with whole chromosome paints according to the Manufacturer's instructions (Cambio, Cambridge, UK). In outline, metaphases were prepared by culturing tumour cells for 4 hours in the presence of 0.2 μg/ml colcemid (Invitrogen). Cells were harvested and resuspended in hypotonic (75 mM) KCl, and incubated for 30 min. at 37° C. Nuclei were washed twice in fixative (3:1 ethanol:glacial acetic acid), re-suspended in fixative and dropped onto glass slides. After denaturation with 70% formamide, metaphase spreads were hybridised with FITC-labelled chromosome 9 paint and Cy3-labelled chromosome 17 paint for 16 hours at 42° C. After 2 stringent washes with 50% formamide, spreads were mounted with DAPI (Vector Laboratories, Burlingame, Calif.) and analysed at 1000x magnification under oil immersion. Image analysis was performed with Smart Capture 2.1 (Digital Scientific, Cambridge, UK). Bone marrow and spleen single cell suspensions were short-term cultured, as for tumour cells for 2-5 days prior to FISH analysis.

### EXAMPLE 1

### Inter-Chromosomal Translocations Between Mouse Mll and Enl Genes

The strategy used to generate chromosomal translocations was to introduce loxP sites into equivalent introns of the Mll and Enl genes to those involved in human leukaemia translocations, using homologous recombination in ES cells. The ES cells were used to make mice carrying these genetic alterations and inter-bred with Cre-expressing mice. We have previously described the mouse Mll-loxP gene in embryonic stem (ES) cells, at a site corresponding to human translocations (FIG. 1A) (Collins et al., 2000). Similarly, a loxP site was engineered into an Enl gene intron, upstream of exon 2 (FIG. 1B). Somatic recombination between these sites should create a mouse fusion gene equivalent to the MLL-ENL fusion found in human leukaemias with t(11;19) (Ayton and Cleary, 2001).

Initial tests were performed to confirm that translocations were possible between mouse chromosomes 9 and 17 (Mll and Enl chromosomes respectively) by expressing Cre protein in ES cells carrying both the Mll and Enl loxP alleles. After transient expression of Cre protein, genomic DNA and mRNA were isolated and recombination products were analysed by genomic PCR or RNA based RT-PCR (FIG. 1C). A genomic PCR product, spanning the Mll-Enl chromosome (t(9;17) junction, was obtained which had the predicted sequence comprising chromosome 9-loxP site-chromosome 17 (FIG. 1D) and the cells were expressing a fusion mRNA, comprising Mll exon 10 fused to Enl exon 2 (Fig. 1E). These data confirm that the t(9;17) Mll-Enl chromosomal translocation can occur in mouse cells and that the fusion mRNA is transcribed.

### EXAMPLE 2

### Myeloid Leukaemias Develop Carrying t(9;17) in Mice

The possible tumourigenic effect of the Mll-Enl chromosomal translocation in mice was determined by studying mice in which recombination between the Mll and Enl genes was mediated by Cre recombinase expressed from a knock-in of Cre into the haematopoietic regulator Lmo2 (manuscript in preparation). A cohort of 21 mice was generated carrying the Mll-loxP and Enl-loxP alleles together with the Lmo2-Cre allele. Mice carrying the two loxP alleles and the Cre allele (Mll-loxP; Enl-loxP; Cre) were compared with mice carrying only the loxP alleles (Mll-loxP; Enl-loxP). By 104 days, 98% of the Mll-loxP; Enl-loxP, Cre mice had died or been sacrificed due to ill health, while all Mll-loxP; Enl-loxP mice remained healthy (FIG. 2A; a similar cohort of Lmo2-Cre-only mice also remained disease-free). Post-mortem examination of Mll-loxP; Enl-loxP; Cre mice showed splenomegaly, pale livers, kidneys and bone marrows. Invasion of spleen was observed, with disruption of normal architecture by leukaemic cells and prominent peri-vascular deposits of tumours cells in liver (FIG. 2B). Blood smears showed large numbers of circulating myeloid cells (FIG. 2B), with a mixture of immature myeloblasts and more mature myeloid forms. The surface antigen phenotype of bone marrow, spleen or thymus cells of tumour mice showed that the Mll-loxP; Enl-loxP; Cre mice develop a leukaemia characterised by expression of the myeloid markers Mac-1 (CD11b) and Gr-1 (Ly-6G) (FIG. 2C). Cell lines, established from spleens of leukaemic mice also have a Mac-1; Gr-1 phenotype, similar to the primary biopsy material (FIG. 2C). The myeloid markers are similar to cultured cells from leukaemic Mll-AF9 knock-in mice (Dobson et al., 1999) (FIG. 2C). We conclude that the Mll-loxP; Enl-loxP; Cre mice develop an acute leukaemia involving myeloid lineage. According to mouse leukaemia classification (Kogan et al., 2002), the disease in these animals could be described as myeloid leukaemia with maturation.

The leukaemia observed in the Mll-Enl mice causes death in all cases, unless the mouse was sacrificed due to obvious ill-heath, and Mll-loxP; Enl-loxP; Cre mice all succumb in the first 120 days after birth. The disease was only observed in individuals with both the loxP alleles and expressing Cre recombinase. This suggested that the cause of leukaemia was the generation of cells in the individual mice with chromosomal translocations which outgrow and present as overt leukaemia. The presence of the reciprocal chromosomal translocations in mice which succumbed to leukaemia was confirmed by analysis of genomic DNA and by direct observation of translocated chromosomes. The presence of translocations in the leukaemias was shown using filter hybridisation of DNA from tumour cells. DNA probes from 5′ or 3′ of the Mll gene breakpoint or from 3′ of the Enl breakpoint were used to examine rearranged genomic DNA restriction fragments (FIG. 3A). This substantiated the presence of reciprocal translocations in the mice, since the anticipated restriction fragment products of translocations were found. These were specifically, a 7.0 Kb SphI fragment detected by the 5′ Mll probe, a 9.5 Kb KpnI fragment detected by the 3′ Mll probe and a 3.0 Kb HindIII fragment detected by the 3′ Enl probe. Formal confirmation of the genomic breakpoint of the translocation was obtained by genomic PCR of a product spanning relevant Mll and Enl introns, followed by DNA sequencing. The presence of the translocated Mll DNA fragment was confirmed in all the leukaemic mice in the cohort. The cloned PCR product from Mll-loxP; Enl-loxP; Cre mouse tumour cells confirmed the site-specific joining of Mll and Enl introns (FIG. 4C), which was identical to that of the ES cell product, and showed site specific recombination between the loxP sites.

A direct visualisation of the reciprocal translocations was obtained by fluorescence in situ hybridisation (FISH) of tumour cell chromosomes from the Mll-loxP; Enl-loxP; Cre mice compared with Mll-loxP; Enl-loxP mice (FIG. 3B). Short term bone marrow cultures were used for the preparation of metaphase chromosomes. This revealed reciprocal chromosomal translocations by hybridisation with chromosome 9 and chromosome 17 paints (FIG. 3B). In addition, metaphases from Mll-loxP; Enl-loxP; Cre tumour cell lines obtained from spleen cells also had reciprocal translocations t(9;17) and t(17;9) (FIG. 3B). The tumours from the Mll-loxP; Enl-loxP; Cre mice therefore carry reciprocal chromosomal translocations.

Chromosomal Translocations Occur Early in Mll-Enl Mice

The youngest Mll-loxP; Enl-loxP; Cre mouse to develop leukaemia was about 1.5 months of age. Thus, the onset of disease in these Mll-Enl translocator mice occurred very early in life and with a high penetrance, and was always associated with the presence of the chromosomal translocations. Therefore, the chromosomal translocations events between Mll and Enl must occur early in the Mll-Enl-Cre mice, to allow time for disease manifestation. Possible timing and frequency of translocations were obtained using FISH on chromosomes from bone marrow cells of young mice (12 days of age). Six Mll-loxP; Enl-loxP; Cre pups from one litter (of 12) were analysed. One showed translocations by FISH analysis in all metaphases examined (FIG. 4A, pup 7, 23 metaphases examined) whilst the other five pups had only normal chromosomes in 20-23 metaphases examined (FIG. 4A; a representative metaphase from pup 10).

Even though we obtained differential results with detection of chromosomal translocations in the 12 day mice, the early onset of leukaemia in the cohort (FIG. 2) was suggestive of early and frequent inter-chromosomal translocations, followed by selection of the cell(s) carrying the aberrant chromosomes. The sensitivity of the analysis for the 12 day pup 7 and pup 10 littermates was, therefore, extended using genomic PCR to detect the junction of the chromosomal translocation t(9;17). DNA was prepared from bone marrow (BM; FIG. 4B) or spleen cells, and splenic DNA from an leukaemic mouse, and quality control was carried out using an Lmo2 gene primer pair. This yielded an Lmo2 product at 30 PCR cycles in all samples. When the Mll-Enl chromosomal junction PCR was assessed at 30 cycles, we detected a product in the DNA of pup 7 bone marrow and the tumour DNA but not in bone marrow DNA of pup 10 nor in either pups 7 or 10 spleen DNA (FIG. 4B, central panel). No product was obtained with DNA from pup 2, which was Mll-loxP; Enl-loxP only. The sequence of the tumour PCR product was obtained to confirm the specific junctional information (FIG. 4C).

The presence of low concentrations of chromosomal translocation-positive cells was assessed in these samples using nested PCR, carried out for a further 30 PCR cycles. In this analysis, we observed translocation-specific PCR products from bone marrow DNA from pup 10 (pup 7 shows a product at 30 cycles) and from spleen DNA of both pups 7 and 10 (FIG. 4B) which did not amplify a product at 30 cycles. No product was obtained from the Cre-negative pup 2. These data therefore suggest that Mll-Enl chromosomal translocations are occurring in young mice, initially in cells of bone marrow origin and subsequently cells with the chromosomal translocation migrate out to spleen.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

1. A method for generating a non-human animal model of a chromosomal rearrangement, comprising creating a transgenic non-human mammal expressing a site-specific recombinase under the control of a cell type-specific promoter, and having sites recognised by the recombinase present in its genome such that a chromosomal rearrangement is catalysed by the recombinase.

2. A method according to claim 1, wherein the chromosomal rearrangement is a translocation.

3. A method according to claim 2, wherein the translocation is a reciprocal translocation.

4. A method according to claim 5, wherein the site-specific recombinase is selected from the group consisting of Cre, Flp and R.

5. A method according to claim 4, wherein the Cre recombinase is combined with loxP sites, the Flp recombinase is combined with frt sites, and/or R recombinase is combined with Rs sites.

6. A method according to claim 1, wherein the chromosomal rearrrangement is tumourigenic.

7. A method according to claim 6, wherein the tumour is a haematopoietic tumour.

8. A method according to claim 7, wherein the cell type-specific promoter is the lmo2 promoter.

9. A method according to claim 7, wherein the tumour is a leukaemia.

10. A method according to claim 9, wherein the sites recognized by the recombinase are located such as to cause the recombination of Mll and Enl genes.

11. A non-human animal tumour model with a chromosomal rearrangement, said animal model expressing a site-specific recombinase under the control of a cell type-specific promoter.

12. A non-human animal model according to claim 11, wherein the chromosomal rearrangement is a translocation.

13. A non-human animal model according to claim 12, wherein the chromosomal translocation is a reciprocal translocation.

14. A non-human animal model according to claim 13, wherein the cell type-specific promoter is an lmo2 promoter.

15. A non-human animal model of leukaemia, which has an Mll-Enl fusion.

16. A non-human animal according to claim 15, which has an Mll-LoxP; Enl-LoxP; Cre genotype.

17. A non-human animal model according to claims 11 or 15, which has leukaemia.

18. A non-human animal model according to claim 17, which is free from secondary mutations.

19. A non-human animal according to claim 17, wherein the leukaemia is myeloid leukaemia.

20. A non-human animal according to claim 19, wherein the leukaemia is myeloid leukaemia having a Mac-1; Gr-1 phenotype.


SEQUENCE LISTING (5 sequences)

SEQ ID NO: 1 - DNA, 439 bp, Artificial
  Genomic PCR fragment comprising the junction of the Mll chromosome, loxP site, and Enl chromosome
cttctcgtctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccagaagagggcgtcagatctnnnnnnnnnnnnnnnnnnnnnnnnnnnnnnnnnnnnnnnataacttcgtataatgtatgctatacgaagttatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgcttccatgtcc

SEQ ID NO: 2 - DNA, 33 bp, Artificial
  PCR primer MR1
tgctttctctgtgccagcagtgggcatgtagag

SEQ ID NO: 3 - protein, 11 aa, Artificial
  Translation of MR1 primer
CFLCASSGHVE

SEQ ID NO: 4 - DNA, 33 bp, Artificial
  PCR primer ER1
tgcactgtccaggtgaagttagagctggggcac

SEQ ID NO: 5 - protein, 11 aa, Artificial
  Translation of PCR primer ER1
CTVQVKLELGH